(19) United States
(12) Patent Application Publication
(10) Pub. No.: US 2023/0157695 A1
(43) Pub. Date: May 25, 2023

(54) HEMORRHOIDS LIGATION TREATMENT DEVICE

(71) Applicant: Top Minimal Invasive Med. & Tech. (Changzhou) Ltd., Changzhou, Jiangsu Province (CN)

(72) Inventors: Rongwang Pei, Suzhou, Jiangsu Province (CN); Long Cui, Shanghai (CN); Zhaoqiang Wang, Shanghai (CN); Yongxun Ma, Panjin, Liaoning Province (CN); Hongfei Du, Shanghai (CN); Jing Gu, Changzhou, Jiangsu Province (CN); Xiang Zhao, Changzhou, Jiangsu Province (CN)

(21) Appl. No.: 17/777,619

(22) PCT Filed: Feb. 7, 2020
(86) PCT No.: PCT/IB2020/000188

§ 371 Date: May 18, 2022

(51) Int. Cl. A61B 17/12

(52) U.S. Cl. CPC A61B 17/12009; A61B 2018/005

(57) ABSTRACT

A binding device for indicating the amount of negative pressure suction to use in a binding operation to treat hemorrhoids. The device is simple and convenient to use, and allows for more accurate binding operations. The binding device has a shell, a pushing pipe at the shell's front end, and a suction tube in the pushing pipe. A negative pressure device is in the shell. A scale cylinder has a cylinder body divided into two cavities and a piston piece within. The negative pressure device has a suction port in communication with the rear end of the suction tube and connected with one of the cylinder cavities. The cylinder body can move relative to the shell, and the shell has a scale part corresponding to that movement. The binding part is sleeved at the front end of the suction tube.

### BACKGROUND OF THE INVENTION

### 1. Field of the Invention

The present invention relates to medical devices, particularly ligation treatment devices and ferrules, and to the treatment of internal hemorrhoids in clinical operation.

### 2. Description of Relevant Art

In surgically treating hemorrhoids, all of the relaxed superficial vein network in the anal canal are ligated so the hemorrhoids will have no source for a blood supply. Hemorrhoids without a blood supply will disappear and be cured. Compared with using an anastomosis device for the ligation, an anorectal applicator can greatly shorten the operation time, reduce the difficulty of operation and improve the quality of operation. In addition, the use of an anorectal applicator not only facilitates the operation process, but also reduces the bleeding and relieve pain and other symptoms of patients postoperatively. Generally, anorectal applicators have a process of suction by negative pressure. However, the amount of suction is not readily determined and remains a problem to be solved.

### SUMMARY OF THE INVENTION

The present invention provides a binding device for use in hemorrhoid ligation. The device has a shell, a push tube in the front end of the shell, and a suction tube in the push tube. The front end of the suction tube extends outward from the front end of the push tube and has a binding piece sleeved at said front end. The device also has a scale cylinder with a cylinder body and a piston piece. The piston piece is fixedly arranged with the interior of the cylinder body such that the piston piece divides the cylinder body into two cylinder cavities—a first cylinder cavity and a second cylinder cavity. The binding device of the invention further has a negative pressure device inside the shell, having a suction port in communication with the back end of the suction tube, and having an air extraction port in communication with the first cylinder cavity. The cylinder body can move relative to the shell, and the shell comprises a with a scale part corresponding to the movement of the cylinder body.

### BRIEF DESCRIPTION OF THE DRAWINGS

FIG. 1 is a cutaway side view showing the interior of one embodiment of the binder or hemorrhoids ligation treatment device of the present embodiment.

FIG. 2 is a perspective side view of an embodiment of the binder of the invention without a pressure relief button.

FIG. 3 is a side view of one embodiment of the binder of the invention with a pressure relief button.

FIG. 4 is a cutaway end view of a section of the binder of the invention shown in FIG. 3.

FIG. 5 is a top view of the binder in FIG. 1.

FIG. 6 is a side view of one embodiment of the binder of the invention having a marker arranged on the scale part of the binder.

FIG. 7 is a side view of one embodiment of the binder of the invention having a scale part in the form of a dial.

FIG. 8 is a side perspective view of the ferrule in one embodiment of the invention.

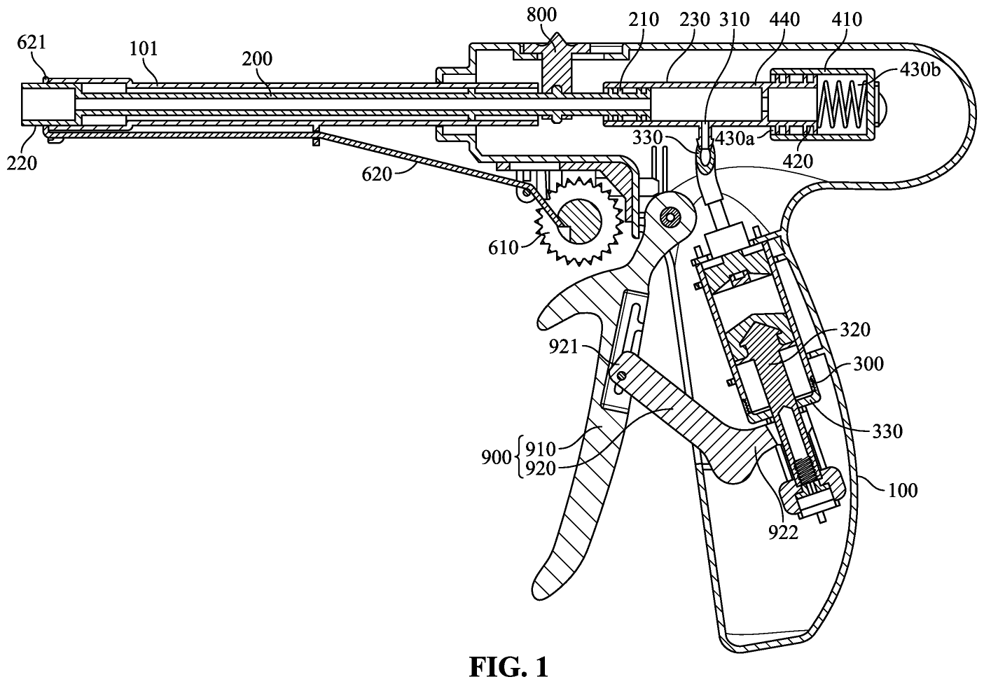

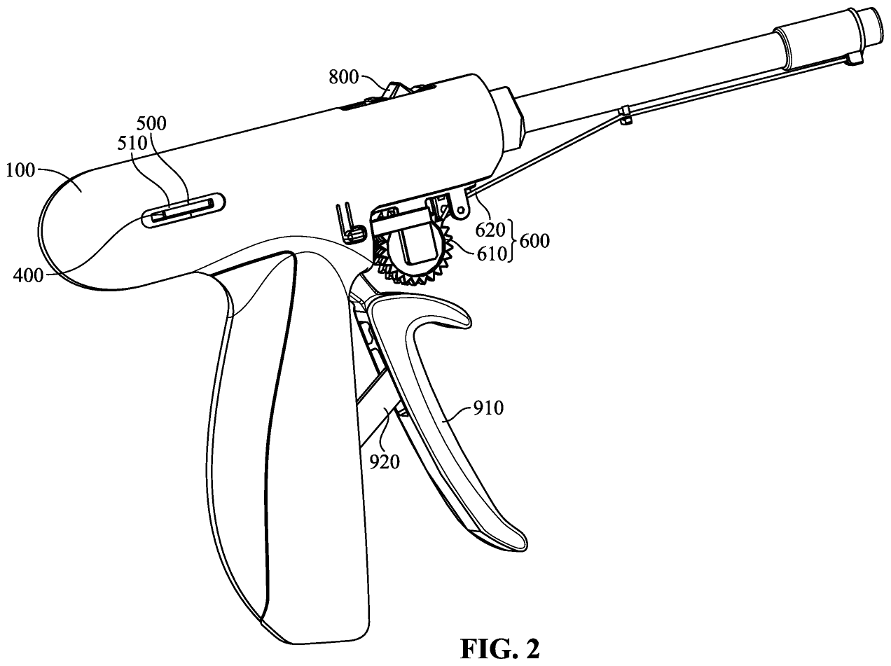

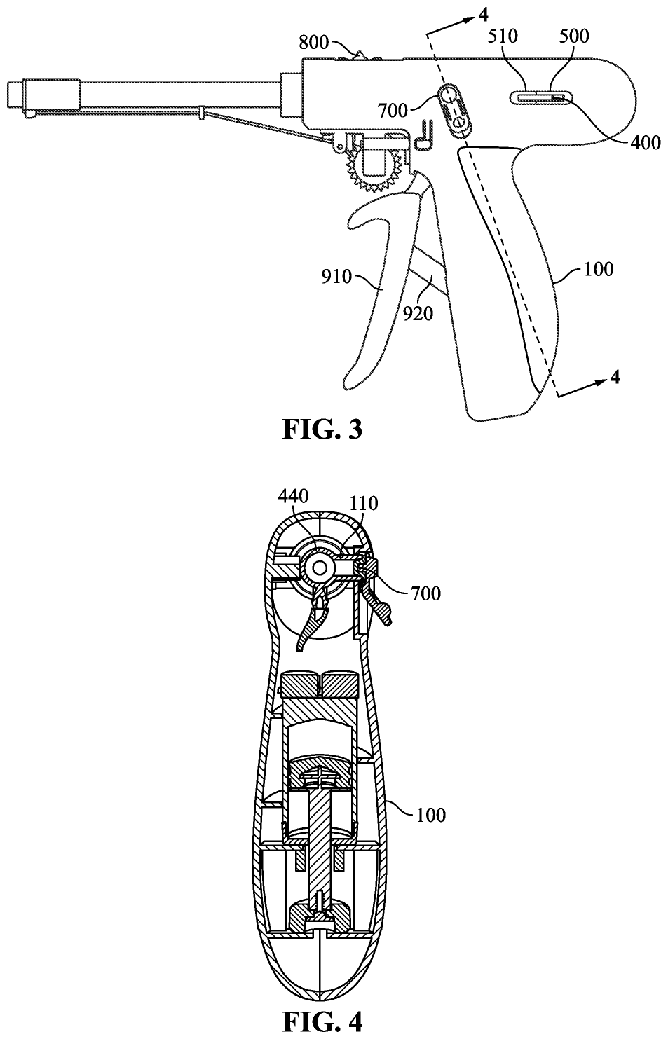

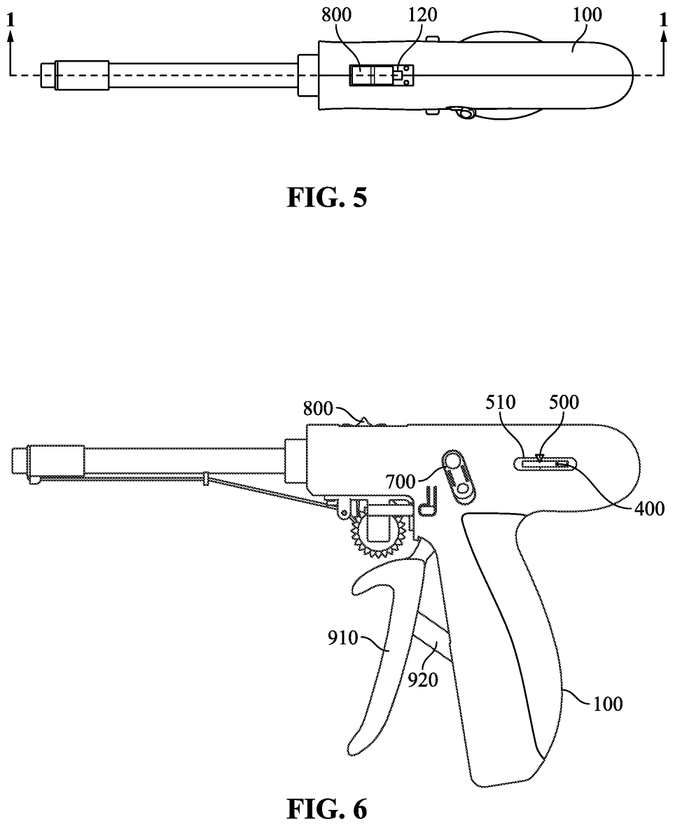

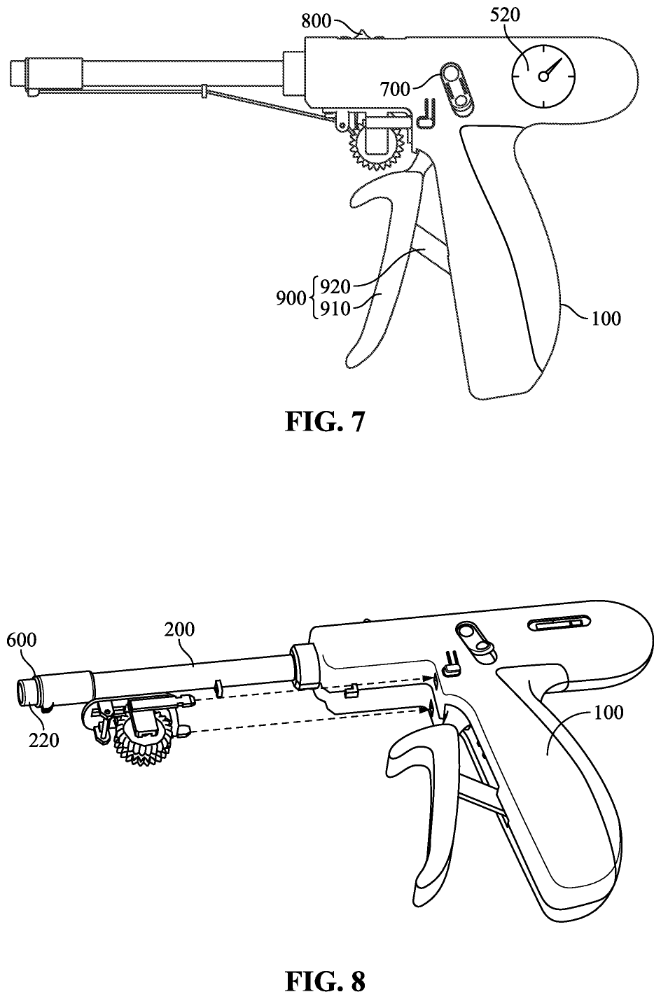

### DETAILED DESCRIPTION

The present invention solves the problems of the prior art associated with indefiniteness in amount of suction to be used with an anorectal applicator. The present invention solves those problems by providing a binding device or binder. Referring to the figures, the binding device of one embodiment of the invention comprises: a shell 100, a push tube 101 at the front end of the shell; a suction tube 200 penetrated within the push tube 101, wherein the front end 220 of the suction tube 200 is penetrated out of the front end of the push tube 101. A negative pressure device 300 is arranged inside the shell, and the negative pressure device 300 has a suction port 310 that is connected with the rear end 230 of the suction tube 200. A scale cylinder 400 is included on the cylinder body 410. The inner part of the cylinder body 410 is provided with a piston piece 420, which is fixedly arranged. The piston piece 420 divides the cylinder body 410 into two cylinder chambers, a first cylinder chamber 430a and a second cylinder chamber 430b and the suction port 310 of the negative pressure device 300 is connected with one of the cylinder chambers—cylinder chamber 430a; the cylinder body 410 can move with the shell 100 relative to each other, and the shell 100 is provided with a scale part 500 corresponding to the moving displacement of the cylinder body 410.

The binding device of the invention is sleeved on the front end 220 of the suction tube 200.

Preferably, the front end of the piston part 420 is connected with the piston rod 440. The piston rod 440 and the piston part 420 are hollow structures. The piston rod 440 is connected with the piston part 420, the piston rod 440 is connected with the suction port 310 of the negative pressure device 300. The two cylinder cavities 430a and 430b are arranged in order from the front [cylinder cavity 430] to the rear [cylinder cavity 430b], and the cylinder cavity 410 at the rear [cylinder cavity 430b] is connected with the piston part 420.

Further, the rear end of the suction tube 200 is inserted into the piston rod 440. Further, the rear end of the suction tube 200 is provided with a convex part 210. The convex part is in the piston rod 440, and the structure of the cross section of the convex part 210 is the same as that of the cross section inside the piston rod 440.

Further, the shell is provided with a gas inlet channel 110, the inlet of the gas inlet channel 110 is provided with a pressure relief button 700, and the outlet of the gas inlet channel 110 is communicated with the piston rod 440.

Further, the binding device also includes a binding release button 800 movably installed on the shell 100, the binding release button 800 can move relative to the shell 100, and the binding release button is connected with the suction tube 200.

In one embodiment, the binding device has a ferrule 620 which has associated with it a take-up device 610. The take-up device 610 is installed on the shell 100; one end of the ferrule 620 is wound on the take-up device 610, the other end of the ferrule is tied with a loose knot to form a ring sleeve 621, and the ring sleeve 621 is tight to the front end 220 of the suction tube 200. Preferably, the ferrule 620 is an elastic ring 600 arranged at the front end 220 of the suction tube 200. Preferably, the ferrule 620 also includes a negative pressure driving component 900, the negative pressure driving component 900 includes a handle 910 which is movably installed on the shell 100, one end 921 of a fork 920 is movably connected with handle 910, the other end 922 of fork 920 is movably connected with the suction piston 320 of the negative pressure device 300.

Preferably, the cylinder body 410 moves along the front and back direction of the binding device; the scale part 500 is a long strip scale bar 510 corresponding to the moving path of the cylinder body 410, or the scale part is a dial 520 corresponding to the moving displacement of the cylinder body 410.

As described above, the ferrule 620 of the invention has the following beneficial effects: When the device of the invention is used, the suction tube 200 of the ligating device is aligned with the hemorrhoid core to be ligated, and after the suction port 310 of the negative pressure device 300 makes the suction action, under the effect of negative pressure, the hemorrhoid core is sucked into the suction tube 200. At the same time, because the piston part 420 is fixedly arranged, the air pressure in the cylinder cavity 410 communicated with the suction port 310 of the negative pressure device 300 is reduced, the cylinder body 410 will move, and the staff will move with the cylinder body through the movement The scale part corresponding to the dynamic displacement is informed of the movement amount of the cylinder body.

When the user of the device of invention is informed by the device that the movement amount of the cylinder body reaches the preset amount, the suction amount of the negative pressure device 300 reaches the preset requirement, and then the user can use the binding parts to bind the hemorrhoids. The present invention allows users of the invention to know the amount of negative pressure extraction, with simple structure, convenient use and more accurate performance of the binding operation.

While preferred embodiments of the present disclosure have been described, it should be understood that other various changes, adaptations, and modifications can be made therein without departing from the spirit of the invention(s) and the scope of the appended claims. The scope of the present disclosure should, therefore, be determined not with reference to the above description, but instead should be determined with reference to the appended claims along with their full scope of equivalents. Furthermore, it should be understood that the appended claims do not necessarily comprise the broadest scope of the invention(s) which the applicant is entitled to claim, or the only manner(s) in which the invention(s) may be claimed.

### APPENDIX: DRAWING LABEL DESCRIPTION

100 shell
101 push tube (or pipe)
110 gas inlet channel
200 suction tube
210 raised part
220 front end of the suction tube 200
230 rear end of the suction tube 200
300 negative pressure device
310 suction port
320 pumping piston
330 trachea
400 scale cylinder
410 cylinder body
420 piston piece
430a first cylinder chamber
430b second cylinder chamber
440 piston rod
500 scale part
510 long strip scale bar
520 dial
600 elastic ring
610 take-up device
620 ferrule
621 ring sleeve
700 pressure relief button
800 binding release button
900 negative pressure driving component
910 handle
920 fork
921 one end of fork 920
922 other end of fork 920

What is claimed is:

1. A binding device for use in hemorrhoid ligation, comprising:
a shell having a front end;
a push tube in the front end of the shell, wherein the push tube has a front end and a back end;
a suction tube penetrated in the push tube, wherein the suction tube has a front end and a back end and the front end of the suction tube is penetrated out of the front end of the push tube and has a binding piece sleeved at said front end;
a scale cylinder having a cylinder body and a piston part fixedly arranged within the interior of said cylinder body such that the piston part divides the cylinder body into a first cylinder cavity and a second cylinder cavity;
a negative pressure device inside the shell, having a suction port in communication with the back end of the suction tube, and having an air extraction port in communication with the first cylinder cavity;
wherein the cylinder body can move relative to the shell, and the shell comprises a scale part corresponding to the displacement movement of the cylinder body.

2. The binding device of claim 1, further comprising a piston rod, wherein:
the front end of the piston part is connected with the piston rod;
the piston rod and the piston part are hollow;
the piston rod is connected with the suction port of the negative pressure device;
the first and second cylinder cavities are arranged in a direction from front to back; and the second cylinder cavity is in communication with the piston piece.

3. The binding device of claim 2, wherein the rear end of the suction tube is inserted into the piston rod.

4. The binding device of claim 3, wherein the rear end of the suction tube has a convex part, the convex part is in the piston rod, and the structure of the cross section of the convex part is the same as that of the cross section of the inner part of the piston rod.

5. The binding device of claim 3, wherein the shell has a gas inlet channel, the inlet of the gas inlet channel has a pressure relief button, and the outlet of the gas inlet channel is in communication with the piston rod.

6. The binding device of claim 2, further comprising a binding release button which is movably installed on the shell, and wherein the binding release button can move relative to the shell, and the binding release button is connected to the suction tube.

7. The binding device of claim 1, further comprising a ferrule with a take-up device, wherein the ferrule has a first end and a second end and the take-up device is installed on the shell such that first end of the ferrule is wound on the take-up device and the second end of the ferrule is tied with a loose knot to form a ring sleeve and the ring sleeve is sleeved tightly on the front end of the suction tube.

8. The binding device of claim 1, further comprising a ferrule that is an elastic ring arranged at the front end of the suction tube.

9. The binding device of claim 1, wherein the negative pressure device has a pressure pumping station, and further comprising a negative pressure driving component having a handle which is movably installed on the shell, a fork having a first and second end with the first end of said fork movably connected to the handle, and the second end of said fork movably connected to the pressure pumping piston of the negative pressure device.

10. The binding device of claim 1, wherein the cylinder body moves along the front and back direction of the binding device; the scale part is a long scale bar corresponding to the moving path of the cylinder body, or the scale part is a scale plate or dial corresponding to the moving displacement of the cylinder body.
